(19) United States
(12) Patent Application Publication
(10) Pub. No.: US 2019/0320891 A1
(43) Pub. Date: Oct. 24, 2019

(54) System, Device and Method for Eye Activity Monitoring

(71) Applicant: Stichting IMEC Nederland, Eindhoven (NL)

(72) Inventors: Alessio Meroni, Eindhoven (NL); Paruthi Pradhapan, Eindhoven (NL); Patrick van der Heijden, Son en Breugel (NL); Navid Shahriari, Weert (NL)

(21) Appl. No.: 16/387,950

(22) Filed: Apr. 18, 2019

(30) Foreign Application Priority Data
Apr. 19, 2018 (EP) 18020165.9

(51) Int. Cl. A61B 3/113; G06F 3/01; A61B 5/0496; A61B 5/00

(52) U.S. Cl. CPC A61B 3/113; G06F 3/015; A61B 5/6814; A61B 5/6803; A61B 5/6819; A61B 5/0496

(57) ABSTRACT

An electronic system, device and method for monitoring eye activity of a subject is provided for monitoring eye activity of a subject using electrooculogram (EOG) biosignals. The electronic system for monitoring eye activity of a subject includes: a plurality of EOG electrodes; electrode circuitry configured for measuring an adapt EOG signals received from the plurality of electrodes; and a data processing unit configured for performing EOG signal processing and eye activity determination; wherein and the data processing unit is configured for performing eye activity determination based on at least a first diagonal eye biosignal vector derived from the biosignals measured between at least a first electrode pair that, in operation, is located on a first diagonal plane around the subject's eye

### CROSS-REFERENCE

This application claims priority from European patent application no. 18020165.9, filed Apr. 19, 2018, which is incorporated by reference in its entirety.

### TECHNICAL FIELD

The present description relates generally to electronic systems for monitoring eye activity of a subject, and more specifically, to an electronic system, device and method for monitoring eye activity of a subject using electrooculogram (EOG) biosignals.

### BACKGROUND

Electrooculography is a methodology for eye-activity monitoring based on the measurement of the electric potential variation on the skin around the eyes due to the rotation of the eyes themselves. This methodology allows measurement of eye-movements and is particularly effective for rapid eye movements (saccades) and blinks.

Traditional EOG measurements consist of the placement of at least four measurement electrodes. In FIG. 1A a conventional electrode configuration for tracking eye activity is shown. This electrode position allows for a measure of the horizontal eye movements through a first pair of electrodes A and D, and vertical eye movements and blinks through a second pair of electrodes B and C. Electrode A can be placed on two locations: as close as possible horizontally next to the eye where electrode D is placed, or on the other side of the face as where electrode D is placed. FIG. 1B shows a vector plot of a known EOG electrode configuration setup corresponding to FIG. 1A. The electrodes A, B, D and C are placed such that the EOG signal activity vectors are orthogonal to each other, thus minimizing cross-talk between the horizontal and vertical eye activity channels.

### SUMMARY

The present description proposes a new and improved electronic system, device and method for eye activity monitoring using EOG biosignal information.

The description can be defined by the claims which include an electronic system for monitoring eye activity of a subject, a wearable device for monitoring eye activity of a subject, and a method for monitoring eye activity of a subject.

Thus in one aspect of the invention, an electronic system (1) for monitoring eye activity of a subject is provided. The electronic system comprises:

a plurality of EOG electrodes (103,A,B,C,D,REF);

electrode circuitry (104) configured for measuring an adapt EOG signals received from the plurality of electrodes (103,A,B,C,D,REF); and

a data processing unit (106) configured for performing EOG signal processing and eye activity determination;

wherein and the data processing unit (106) is configured for performing eye activity determination based on at least a first diagonal eye biosignal vector (Diagonal 1) derived from the biosignals measured between at least a first electrode pair (B,C) that, in operation, is located on a first diagonal plane (D1) around the subject's eye.

In another aspect of the invention, a wearable device (100,200) for monitoring eye activity of a subject is provided. The wearable device, comprises:

a plurality of EOG electrodes (103,A,B,C,D,REF) and electrode circuitry (104) configured for measuring an adapt EOG signals received from the plurality of electrodes;

wherein, the plurality of EOG electrodes (103,A,B,C,D,REF) are configured in electrode measurement pairs, comprising at least a first electrode pair (B,C) that, when the wearable device is placed on the face of the subject, is located on a first diagonal plane (D1) around the subject's eye and the electrode circuitry (104) is configured for measuring the EOG signals between said first electrode pair (B,C).

In another aspect of the invention, a method for monitoring eye activity of a subject is provided. The method comprises:

receiving a plurality of EOG signals from a plurality of electrodes (103,A,B,C,D,REF), said EOG signals being measured between at least a first electrode pair (B,C) that, in operation, is located on a first diagonal plane (D1) around the subject's eye; and

performing EOG signal processing and eye activity determination based on at least a first diagonal eye biosignal vector (Diagonal 1) derived from said biosignals measured between said at least first electrode pair (B,C).

According to an example embodiment, the electronic system for monitoring eye activity of a subject according to the present description allows for a new EOG electrode configuration that can be implemented in a more convenient form factor.

According to an example embodiment, the electronic system for monitoring eye activity of a subject according to the present description, allows for placing the EOG electrodes in locations around the eye that can be easily integrated in standard glasses, smart-glasses and/or smart-goggles.

According to an example embodiment, the electronic system for monitoring eye activity of a subject according to the present description can provide an improved signal-to-noise ratio for signals deriving from the EOG electrodes. The derived EOG biosignals are larger in amplitude with respect to prior art electrode layouts and the SNR is improved, especially when the signals are combined to extract vertical and lateral/horizontal eye movements.

According to an example embodiment, the electrode location allows minimization of the overlap with face muscles in order to minimize the impact of artifacts due to facial expressions.

### BRIEF DESCRIPTION OF THE DRAWINGS

The above and other aspects of the electronic system, device and method according to the present description will be shown and explained with reference to the non-restrictive example embodiments described hereinafter.

FIG. 1A shows a prior art EOG electrode configuration for eye activity measurements.

FIG. 1B shows an eye activity vector plot for a prior art EOG electrode configuration according to FIG. 1A.

FIG. 2 illustrates an EOG electrode location configuration in the face of a subject according to an example embodiment of the present description.

FIG. 3A is an example illustration of an eye activity vector plot for an EOG electrode configuration as in FIG. 2.

FIG. 3B is an example illustration of a vector combination of diagonals to derive the horizontal and vertical eye activity according to an EOG electrode configuration as in FIG. 2.

FIG. 4A is an example flow chart for horizontal and vertical eye-movements determination from two diagonal eye EOG signals.

FIG. 4B is an example flow chart illustrating a method for blink determination and eye spectral/statistical analysis based on a single diagonal eye EOG signal.

FIG. 5 illustrates the function of a data processing unit for eye activity monitoring according to an example embodiment.

FIG. 6 shows a system for eye activity monitoring according to a first example embodiment.

FIG. 7 shows a system for eye activity monitoring according to a second example embodiment.

FIG. 8 shows an electrode circuitry module from a system for eye activity monitoring according to an example embodiment.

FIG. 9 shows a perspective view of a device for eye activity monitoring in the form factor of a pair of glasses according to an example embodiment.

FIG. 10 shows a top view of the device for eye activity monitoring of FIG. 9.

FIG. 11 illustrates a method for measuring EOG signals in a system for eye activity monitoring according to a first example embodiment.

FIG. 12 illustrates a method for measuring EOG signals in a system for eye activity monitoring according to a second example embodiment.

FIG. 13 shows a graph of the horizontal eye activity information derived from diagonal electrode pairs according to an example embodiment compared to a prior art conventional electrode configuration.

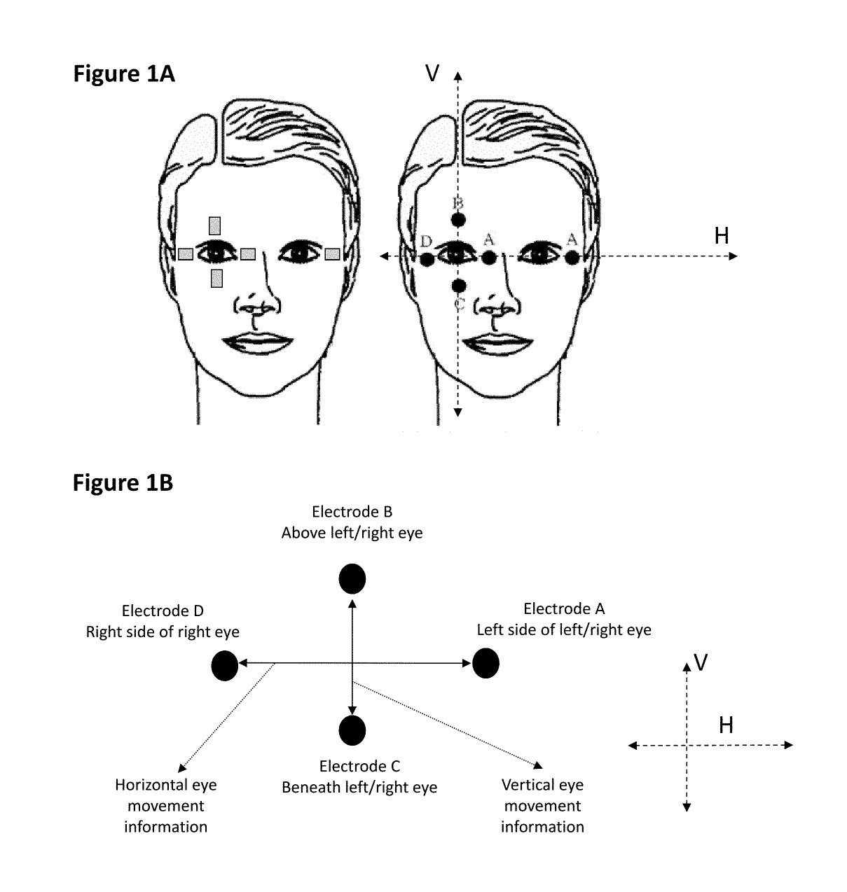

### DETAILED DESCRIPTION

In the following, in the description of example embodiments, various features may be grouped together in a single embodiment, figure, or description thereof for the purpose of streamlining the disclosure and aiding in the understanding of one or more of the various inventive aspects. This is however not to be interpreted as the description requiring more features than the ones expressly recited in the main claim. Furthermore, combinations of features of different embodiments are meant to be within the scope of the description, as would be clearly understood by those skilled in the art. Additionally, in other instances, well-known methods, structures and techniques have not been shown in detail in order not to obscure the conciseness of the description.

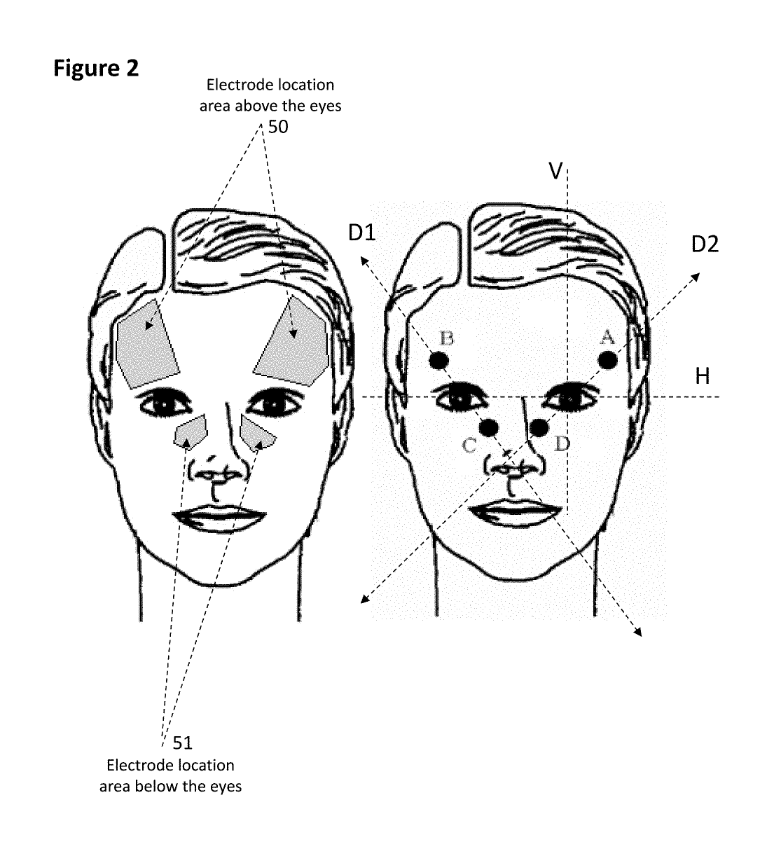

FIG. 2 shows an EOG electrode location configuration in the face of a subject according to an example system according to the present description. According to an example embodiment, two electrodes C and D are placed on the nose or in proximity of the nose below the eyes and another two electrodes A and B are placed in the facial area between temporal and frontalis muscles. According to an example embodiment, the electrodes form diagonal pairs, that is, a first pair of electrodes B and C located on a first diagonal plane D1 around the right eye and a second pair of electrodes A and D located on a second diagonal plane D2 around the left eye. The diagonal planes D1 and D2 in which the electrodes are located, are neither parallel nor perpendicular to the horizontal plane H defined by of the eyes of the subject. The shaded or grey areas 50 and 51 in the figure represent the area of the subject's face that are well-suited for electrode positioning. These shaded areas are close to the eyes, which are needed for detecting the eye activity.

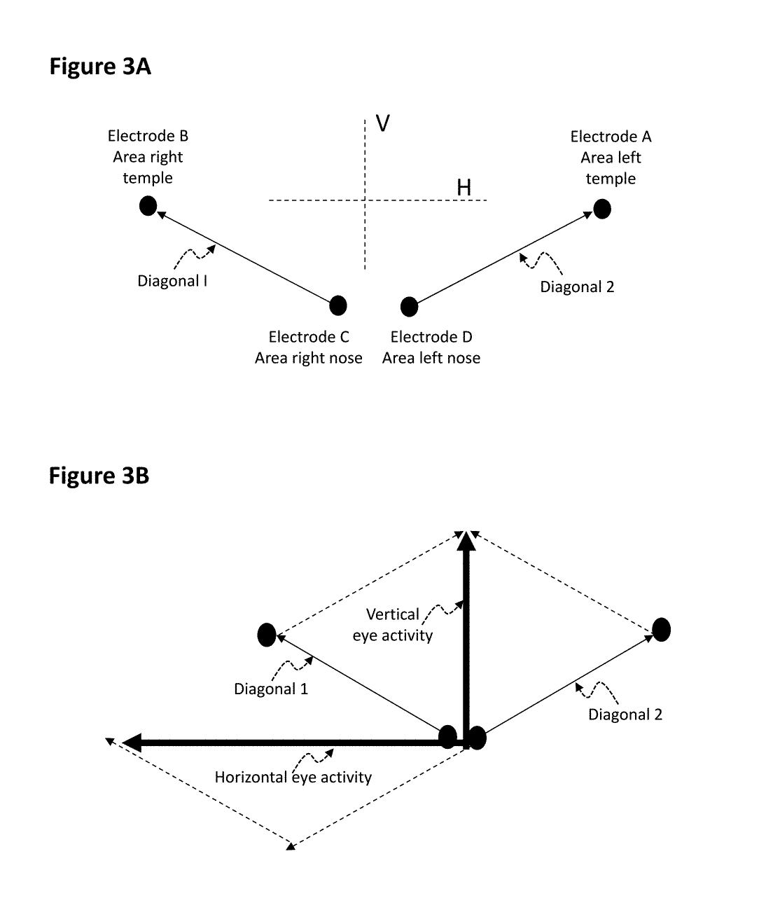

According to an example, the system can be configured to measure the electrical eye activity between the first pair of electrodes B and C and between the second pair of electrodes A and D. These measurements define a first diagonal eye biosignal vector (Diagonal 1 in FIG. 3A) and a second diagonal eye biosignal vector (Diagonal 2 in FIG. 3A). In an embodiment, the location of the electrodes A, B, C, and D may be optimized to minimize the amount of noise originating from the facial muscles which could interfere with the eye activity measurement, and to minimize the amount of skin movements (which cause motion artifacts) at those electrode locations. According to an example embodiment, the electrodes A, B, C, and D are positioned such that muscle and movement artifacts can be minimized, while the signal-of-interest can be maximized. Furthermore, the electrode position can be comfortable for the user and can be adaptable to a widely accepted wearable form factor such as glasses.

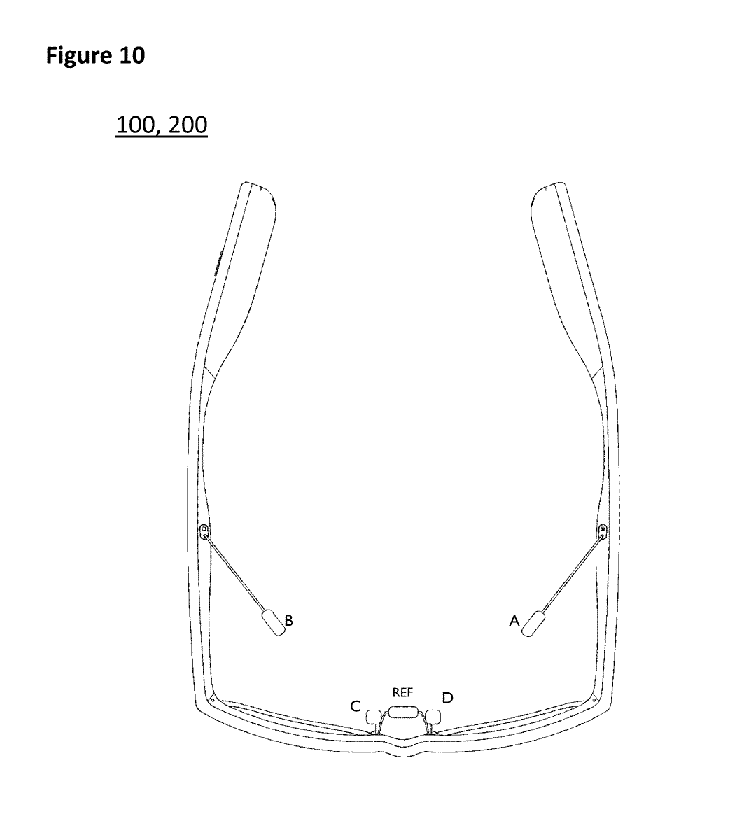

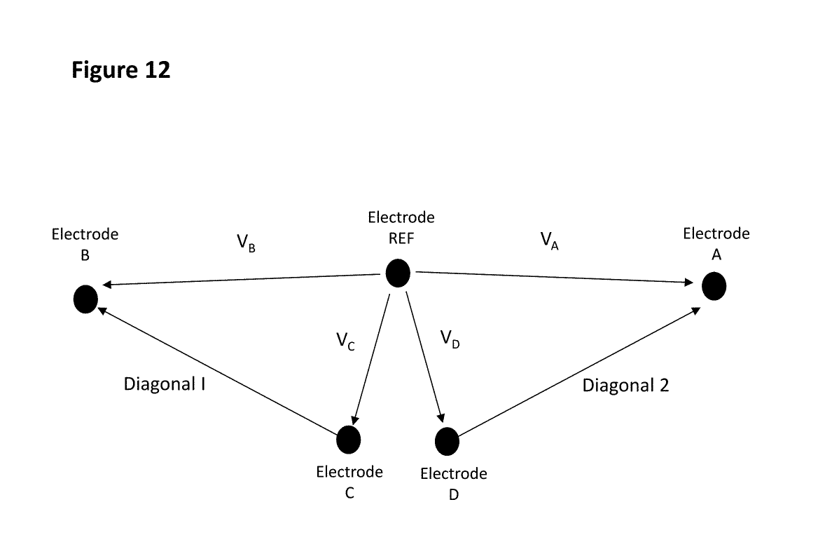

A four-electrode configuration as shown in FIGS. 2, 3, and 11 allows detection of horizontal and vertical eye movements and blinks. According to another embodiment, a five-electrode configuration (comprising a reference electrode, as shown in FIGS. 10 and 12) may be also used for the same purpose. According to another embodiment, a two-electrode configuration (for example one single pair of electrodes B and C or A and D in FIG. 2) may be also used for blink detection and statistical and spectral analysis of eye-activity.

FIG. 3A shows an eye activity signal vector plot for an EOG electrode configuration and measurement according to FIG. 2. According to an example embodiment, a first diagonal vector Diagonal 1 and second diagonal vector Diagonal 2 are measured and determined. The diagonal vectors are neither located in a horizontal plane H nor in a plane orthogonal to that horizontal plane. A horizontal and a vertical eye activity information, including blinks, may be mathematically derived by combining both diagonal vectors Diagonal 1 and Diagonal 2, as shown in FIG. 3B. According to another embodiment, blink information and eye-activity may be also derived from a single diagonal vector Diagonal 1 or Diagonal 2. According to an example embodiment, the horizontal and vertical vectors can be derived from the diagonal EOG signal vectors Diagonal 1 and Diagonal 2 as follows:

EOG Horizontal=Diagonal 1−Diagonal 2

EOG Vertical=Diagonal 1+Diagonal 2

EOG Blink=f (Diagonal 1) or f (Diagonal 2)

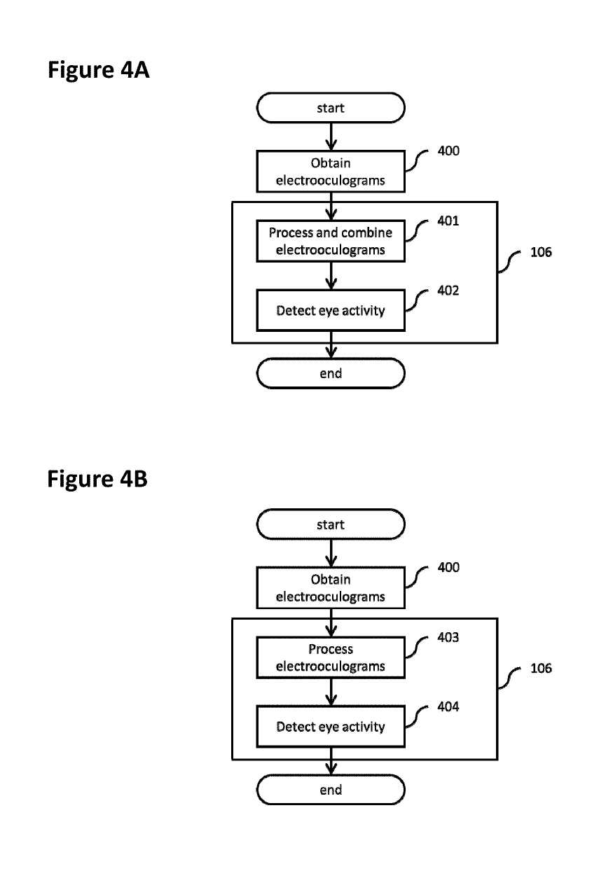

FIG. 4A is an example flow chart for horizontal and vertical eye-movements determination from two diagonal eye EOG signals in a system with electrodes located as in FIG. 2. According to an example embodiment, first the electrical activity of the eyes can be measured 400 by the four EOG electrodes A, B, C, and D in a configuration as shown in FIG. 2. The signals coming from the four electrodes are processed and combined 401 as in FIGS. 3A and 3B, and then used to detect eye-activity 402.

According to another embodiment, shown in FIG. 4B, the electrical activity of an eye can be measured by two diagonal EOG electrodes 400, for example a single pair of electrodes (B and C or A and D). The signals coming from the two electrodes are then processed 403 and used to detect eye-activity 404. The system for eye activity monitoring according to an embodiment, comprises a data processing unit 106 that can be configured for performing EOG signal processing, EOG signal combination and eye activity determination.

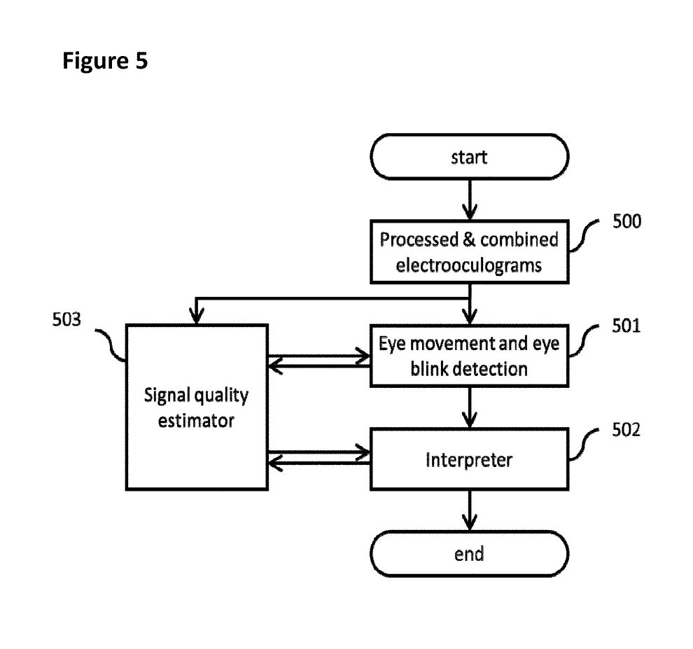

FIG. 5 illustrates the function of a data processing unit 106 for eye activity monitoring according to an example embodiment. In a first step, a first processing module 500 receives EOG biosignal data from the electrodes and associated electronics and, depending on the number of electrodes measured, it processes (in case of a single electrode pair and a single diagonal vector calculation) and/or combines the signals (in case of two electrode pairs and two diagonal vector calculations). An interpreter module 502 interprets the output data from an eye movement and eye blink detection module 501, by combining or discarding or passing-through the detected eye movements and eye blinks. According to an example embodiment, the interpreter module 502 can be configured for different application requirements. A signal quality estimator module 503 receives the EOG signals from the first processing module 500 and estimates the quality of the signal. The signal quality estimator module 503 communicates with the eye movement and eye blink detection module 501 and the interpreter module 502 in order to adapt internal settings. The interpreter module 502 may determine and provide the following information: eye movements information (e.g. type, direction, magnitude, timing, etc.); eye blinks information (e.g. timing, voluntary/involuntary, etc.); and/or eye activity spectral and statistical analysis.

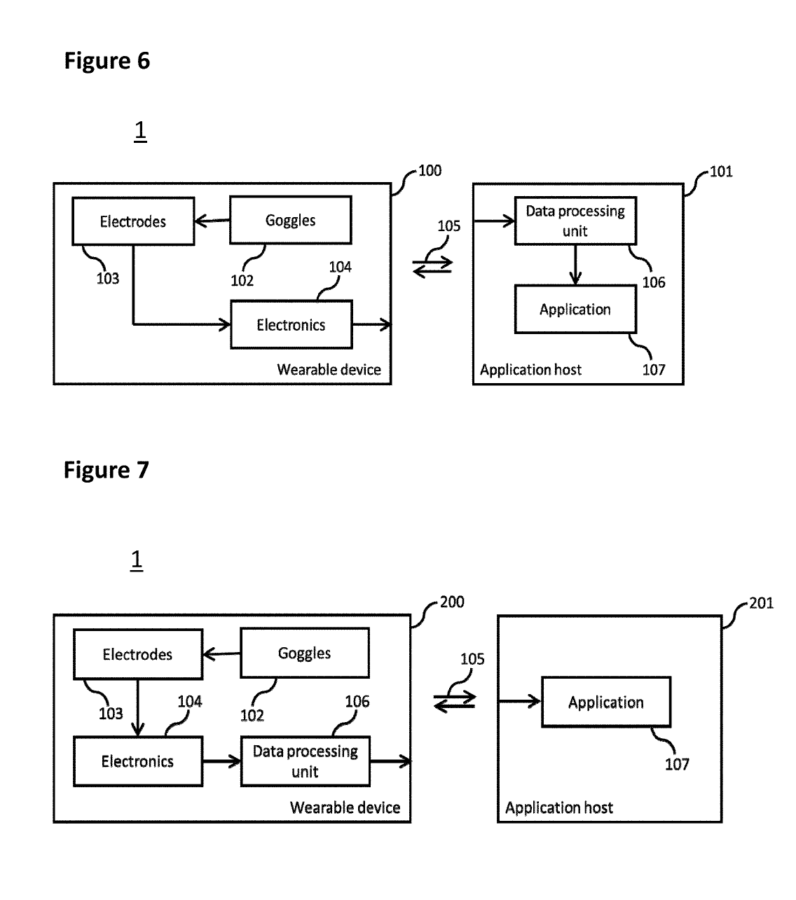

FIG. 6 shows a system 1 for monitoring eye activity of a subject according to a first example embodiment, comprising a plurality of EOG electrodes 103 and associated electrode circuitry 104 integrated in a wearable device 100. The wearable device 100 may comprise glasses, googles, eye lenses or imaging units 102. The wearable device can be worn by the subject on the face and can have the form factor of a pair of glasses with integrated electrodes and associated electrode circuitry. Other form-factors are possible, like augmented (AR), mixed or virtual reality (VR) glasses or display devices. The wearable device 100 can be communicatively coupled, via a wired or wireless connection unit 105, to an application host unit 101. According to an example embodiment, the application host unit 101 comprises a data processing unit 106 as, for example, described in FIG. 5. The data processing unit 106 can be configured then to provide eye activity information to an application module 107.

FIG. 7 shows a system 1 for monitoring eye activity of a subject according to a second example embodiment, comprising a plurality of EOG electrodes 103, associated electrode circuitry 104 and a data processing unit 106 (as, for example, described in FIG. 5), integrated in a wearable device 200. The wearable device 200 may comprise glasses, googles, eye lenses or imaging units 102. The wearable device can be worn by the user on the face and can have the form factor of a pair of glasses with integrated electrodes and associated electrode circuitry. Other form-factors are possible like augmented, mixed or virtual reality glasses or display devices. The wearable device 200 can be communicatively coupled, via a wired or wireless connection unit 105, to an application host unit 201. The application host unit 201 comprises an application module 107 that receives eye activity information from the data processing unit 106.

The data processing unit 106 can be executed on an external computing device (as in FIG. 6) or can be embedded on the wearable device (as in FIG. 7), for example a pair of glasses. According to an embodiment, the wearable device 100, 200 may be implemented and given the form of a pair of glasses or goggles, which will be the wearable structure that holds the electrodes and the electronics in place. The application host 101,201 may be a physical device or a cloud service.

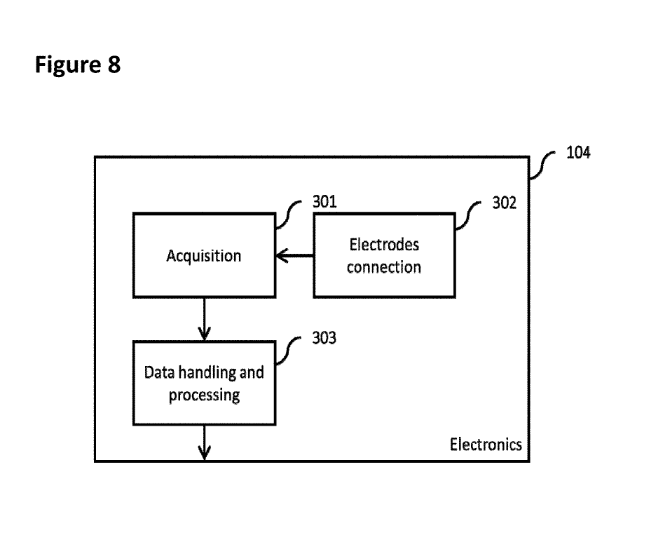

FIG. 8 shows an example electrode circuitry module 104 from a system for eye activity monitoring as shown in FIGS. 6 and 7, comprising an EOG signal acquisition electronic unit 301, an electrode connection unit 302 and an EOG signal handling and processing unit 303. The electrode circuitry module 104 may be integrated in the wearable device 100, 200. Eye activity signals are captured by EOG signal acquisition electronic unit 301. The EOG signal handling and processing 303 treats the signals and data and passes it to either the data processing unit 106 or to the connection unit 105.

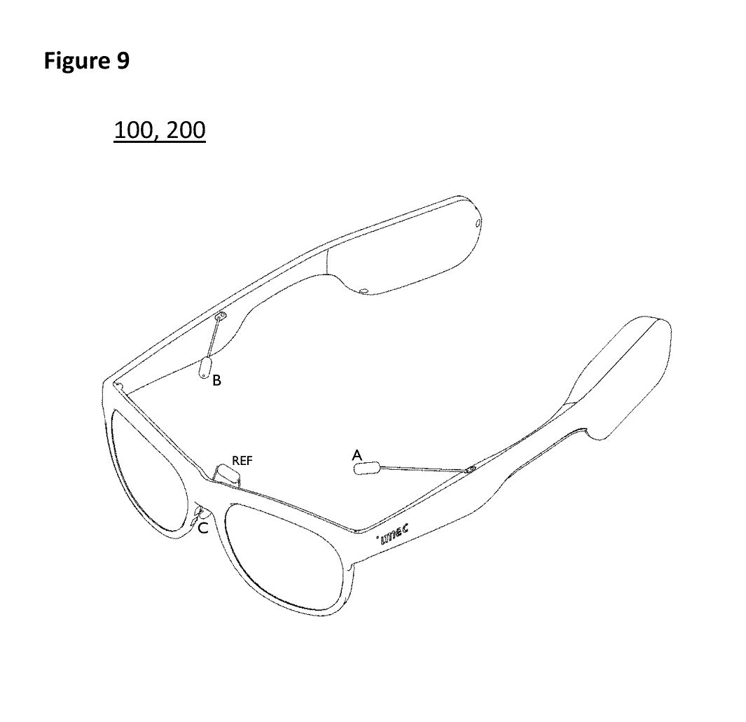

FIG. 9 shows a perspective view of an example wearable device 100, 200 for eye activity monitoring in the form factor of a pair of glasses. The wearable device 100, 200 comprises a plurality of EOG electrodes 103, associated electrode circuitry 104 and may even comprise a data processing unit 106 as in FIGS. 6 and 7. The wearable device 100, 200 may acquire, determine and provide eye activity information based on a two-electrode configuration (single diagonal electrode pair), a four-electrode configuration (two diagonal electrode pairs) and/or a five-electrode configuration (two diagonal electrode pairs plus a reference electrode).

In an embodiment, one, two or three EOG electrodes (electrodes C, D and REF as shown in FIG. 10) may be located in the region of the wearable structure touching the nose of the subject. Two additional electrodes A and B may be also linked to the wearable device and configured for touching or being placed in the facial area of the subject between the temporal and frontalis muscles. It shall be noted that the wearable device 100, 200 of FIGS. 9 and 10 are an example embodiment and other implementation form factors and designs are possible. For example, according to an embodiment, the wearable device 100, 200 may be implemented in the form factor of AR, VR goggles or VR display devices and the EOG electrodes may be integrated in the foam mask of the wearable device.

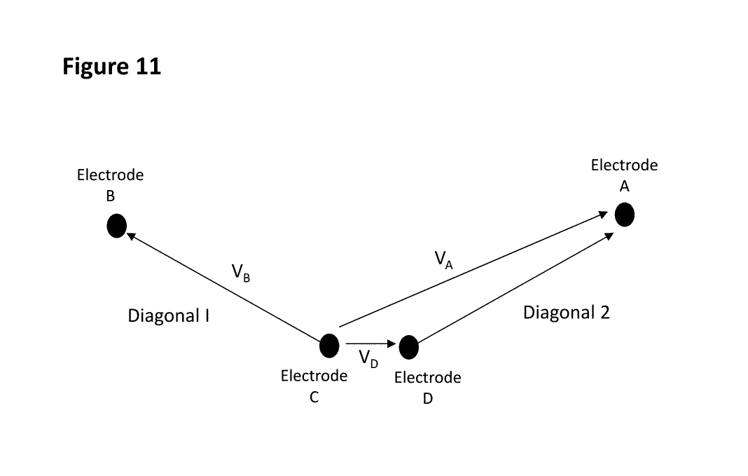

FIG. 11 illustrates a method for measuring EOG signals in a system for monitoring eye activity of a subject according to a first example embodiment, comprising four EOG electrodes A,B,C and D an in which one of the electrodes can be a reference electrode. According to an embodiment, in order to measure the electrical potential on the electrodes in position A,B,C and D, one electrode can be used as reference, and the EOG diagonals vectors can be derived as combination of the other potentials:

VREF=V_{C}

Diagonal 1=V_{B}

Diagonal 2=V_{A}−V_{D}

FIG. 12 illustrates a method for measuring EOG signals in a system for eye activity monitoring according to a second example embodiment, comprising five EOG electrodes A, B, C, D and REF an in which one of the electrodes REF can be a reference electrode. According to an embodiment, the reference electrode REF can be placed anywhere in the face of the user. According to example embodiments, the reference electrode REF may be placed in the forehead of the subject, or between electrodes A and B, or in the top of the nose area between the eyes. In this electrode configuration, the EOG diagonal vectors can be derived as follows:

Diagonal 1=V_{B}−V_{C}

Diagonal 2=V_{A}−V_{D}

According to an example embodiment, the wearable device 100, 200 of FIGS. 9, 10 can be implemented using a five-electrode configuration as described in FIG. 12, comprising: a reference electrode REF placed in the middle of the nose slightly above the eyes, a first electrode A placed at left temple area, a second electrode B placed at right temple area; a third electrode C placed at the right nose area, and a fourth electrode D placed at left nose area. The EOG activity can be measured between the reference electrode REF and all the other electrodes. Thus, four eye signals are measured. The two diagonal vectors can be derived by combining these 4 signals by addition and subtraction.

It shall be noted that the reference electrode location can be changed to any other location on the face and the body of the user. However, according to embodiment of FIGS. 9 and 10, the reference electrode REF location may be very well suited for good skin contact using the current form factor of the goggles, placed in the middle of the nose slightly above the eyes. Other form factors could change the reference electrode location. It is even possible to replace the reference electrode with one of the other electrodes (at temple or nose area) without losing functionality of the diagonals as described in this document. Therefore, in another embodiment, four electrodes could be used (1 at left temple area, 1 at right temple area, 1 at left nose area, 1 at right nose area), with 1 electrode being the reference electrode, and the diagonals being derived from the four activity measurements.

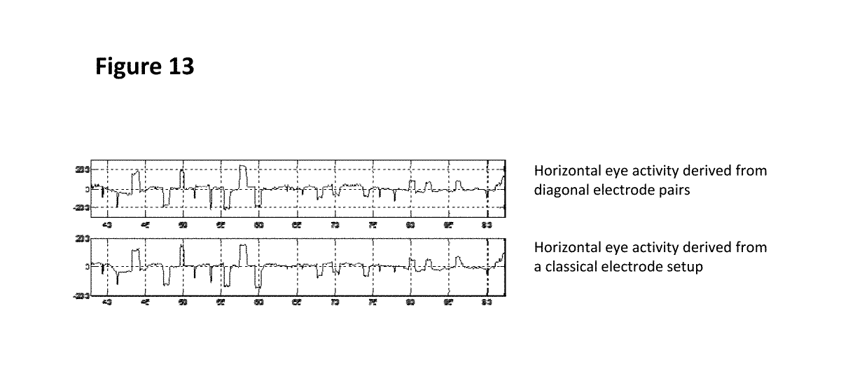

FIG. 13 shows a graph of the horizontal eye activity information derived from diagonal electrode pairs according to an example embodiment compared to a prior art electrode configuration. The top plot shows the horizontal eye activity from the determined diagonal channels according to the current description and the bottom plot the horizontal eye activity derived from a classical EOG electrode setup. The top plot shows the horizontal electrical eye activity measured after combining the two diagonal measurements. The Signal-to-Noise ratio of the diagonal configuration can be larger than the SNR of the classical EOG electrode configuration setup.

It shall be noted that the system 1 system for monitoring eye activity of a subject according to embodiments herein described may comprise units and modules that may be implemented according to hardware and/or software state of the art techniques, comprising for example a microprocessor, microcontroller or digital signal processor that can understand and execute software program instructions. Some programmable hardware logic and memory means may be specifically designed also for executing the signal processing methods or parts of it according to exemplary embodiments. It is also understood that the units of the system described may be implemented in distributed locations, for example in a mobile device and/or a network. Any storage units needed may be implemented as a unique storage memory and/or a distributed memory.

1. An electronic system for monitoring eye activity of a subject, comprising:
a plurality of electrooculogram (EOG) electrodes;
electrode circuitry configured for measuring an adapt EOG signals received from the plurality of electrodes; and
a data processing unit configured for performing EOG signal processing and eye activity determination;
wherein and the data processing unit is configured for performing eye activity determination based on at least a first diagonal eye biosignal vector derived from biosignals measured between at least a first electrode pair that, in operation, is located on a first diagonal plane around the subject's eye.

2. An electronic system according to claim 1, wherein the first electrode pair comprises a first electrode located in the facial area of the subject between temporal and frontalis muscles and a second electrode located on the nose below the eyes of the subject.

3. An electronic system according to claim 1, wherein the first diagonal plane around the subject's eye is neither parallel nor perpendicular to a horizontal plane defined by the eyes of the subject.

4. A wearable device for monitoring eye activity of a subject, comprising:
a plurality of EOG electrodes and electrode circuitry configured for measuring an adapt EOG signals received from the plurality of electrodes;
wherein, the plurality of EOG electrodes are configured in electrode measurement pairs, comprising at least a first electrode pair that, when the wearable device is placed on the face of the subject, is located on a first diagonal plane around the subject's eye and the electrode circuitry is configured for measuring the EOG signals between said first electrode pair.

5. A wearable device according to claim 4, wherein a first electrode pair comprises a first electrode configured for touching the facial area of the subject between temporal and frontalis muscles and a second electrode configured for touching the nose below the eyes of the subject.

6. The wearable device according to claim 4, wherein the at least first electrode pair is integrated in a pair of googles, eye lenses or imaging units.

7. The wearable device according to claim 4, wherein the first diagonal plane around the subject's eye is neither parallel nor perpendicular to a horizontal plane defined by the eyes of the subject.

8. The wearable device according to claim 4, wherein the wearable device is communicatively coupled via a wired or wireless connection unit to an application host unit comprising a data processing unit.

9. The wearable device according to claim 8, wherein the data processing unit is configured for performing eye activity determination based on at least a first diagonal eye biosignal vector derived from biosignals measured between at least a first electrode pair that, in operation, is located on a first diagonal plane around the subject's eye.

10. A method for monitoring eye activity of a subject, comprising:
receiving a plurality of EOG signals from a plurality of electrodes, said EOG signals being measured between at least a first electrode pair that, in operation, is located on a first diagonal plane around the subject's eye; and
performing EOG signal processing and eye activity determination based on at least a first diagonal eye biosignal vector derived from said biosignals measured between said at least first electrode pair.
